(19) United States
(12) Patent Application Publication
(10) Pub. No.: US 2009/0204180 A1
(43) Pub. Date: Aug. 13, 2009

(54) System for implanting a microstimulator

(76) Inventor: Daniel Gelbart, Vancouver (CA)

Correspondence Address: DANIEL GELBART, 4706 DRUMMOND DR, VANCOUVER, BC, V6T-1B4, CA

(21) Appl. No.: 12/068,878

(22) Filed: Feb. 13, 2008

(51) Int. Cl. A61N 1/05

(52) U.S. Cl. 607/61

(57) ABSTRACT

A system for implanting a microstimulator uses an insulated electrical conductor connected to an electrical stimulator and fed through a metal hypodermic needle to locate the best position for stimulation, followed by the insertion of a metal encased micro stimulator.

### FIELD OF THE INVENTION

The invention relates to the medical field and more specifically to electronic muscle and neural stimulation.

### BACKGROUND OF THE INVENTION

It is well known that human muscles and nerves can be stimulated by using electrical pulses. The stimulation can be done by at least five different methods: by attaching external electrodes, by implanting internal electrodes, by wireless internal electrodes, by electrodes using an internal power source or by inducing a voltage inside the body without the use of electrodes. While the last method seems the most attractive it is not practical for many applications as it requires magnetic fields of about 1 Tesla. Such fields are difficult to achieve without close proximity to a large coil. By comparison, wireless internal electrodes can be made to work with fields under 0.001 Tesla. Stimulators using an internal battery require battery replacement surgery and take up significant space. This is justified for some applications, such as pacemakers, but not for less life-threatening situations. Methods requiring wires, either connected to internal electrodes or external electrodes, are less convenient. A particularly convenient method is stimulation using a microstimulator that can be implanted without surgery. A well known microstimulator is the BION™ implant described in U.S. Pat. No. 5,312,439, hereby incorporated by reference. The BION is a miniaturized implantable electrode complete with a pick-up coil and pulse generator. The small size of the BION, about 2 mm in diameter, allows delivery via a hypodermic needle without surgery. The delivery of a BION is typically performed via a large hypodermic needle made of an insulating material, as disclosed in U.S. Pat. No. 6,214,032, hereby incorporated by reference. The microstimulator is powered by an external transmitter having a large coil and operating typically at a frequency in the range 200 KHz to 1 MHz. The details of microstimulators and transmitters are well known in the art and will not be further detailed in this disclosure. It is desired to stimulate the tissue with a removable electrode before the final implantation of the microstimulator is done, in order to optimize the placement. The prior art, shown in FIG. 1, used an insulating hypodermic needle 1, large enough to accommodate the microstimulator. After the needle is positioned in the tissue 4, a metal wire 2 is inserted via the needle and connected to an electrical stimulator 3. By moving the needle, the best place for stimulation is found. The metal wire is removed and the microstimulator is inserted via the needle until it reaches the same position as the tip of metal wire 2. As the needle is electrically non-conductive, the microstimulator can be tested before the needle is removed. One disadvantage of the prior art systems is the fact that the microstimulator enclosure is made of glass or ceramic, therefore can shatter. To protect it from shattering a silicone coating can be used; however this increases the diameter of the micro stimulator.

A second disadvantage of the prior art is the fact the insertion needle is made of an insulating polymer. Polymers are significantly weaker than metals, thus the wall thickness of a polymer needle is considerably higher than a metal needle of equal strength. The combination of these two factors requires a needle with an outside diameter of about 3 mm. One object of the invention is to enable the use of significantly smaller needles for injecting microstimulators.

A third disadvantage of prior art is the additional space taken up by the stimulation electrodes. One object of the invention is to use the microstimulator case as electrodes, thus decreasing the size of the device. Another object is to use a combination of a metal needle and a metal encased microstimulator to further reduce the diameter of the injection needle. A further object of the invention is to use two electrodes for stimulation when locating best placement sites, as two electrodes better emulate the microstimulator. Further objects and advantages will become apparent from the disclosure and the drawings.

### SUMMARY OF THE INVENTION

A system for implanting a microstimulator uses an insulated electrical conductor connected to an electrical stimulator and fed through a metal hypodermic needle to locate the best position for stimulation, followed by the insertion of a metal encased micro stimulator.

### BRIEF DESCRIPTION OF THE DRAWINGS

FIG. 1 is a cross section of the prior art.

FIG. 2 is a cross section of using a metallic needle and an insulated conductor according to the invention.

FIG. 3 is a cross section of using a metallic needle and two insulated conductors according to the invention.

FIG. 4 is a cross section of a needle implanting a microstimulator according to the invention.

FIG. 5 is a cross section of a microstimulator using a metallic case.

FIG. 6 is an alternate method of hermetically sealing a metallic case of a micro stimulator.

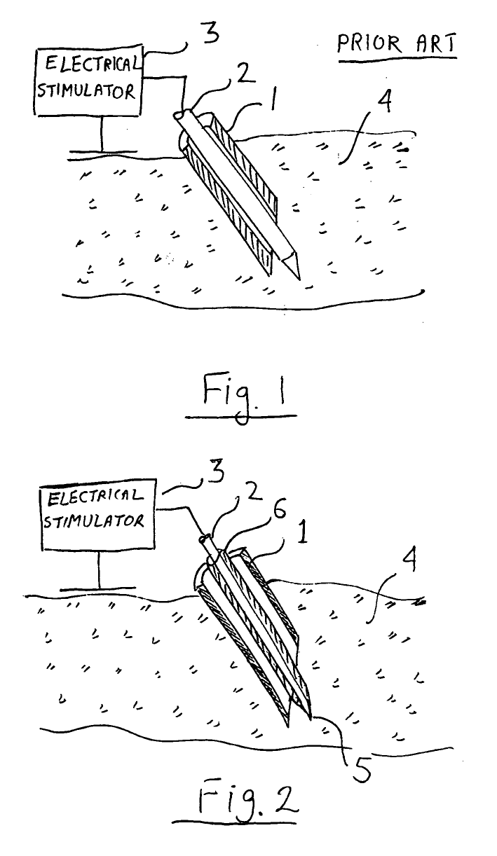

### DESCRIPTION OF THE PREFERRED EMBODIMENT

FIG. 2 shows a cross section of a regular metallic hypodermic needle 1 inserted in tissue 4. A metal wire 2 insulated by insulation 6 is connected to electrical stimulator 3 and stimulates the tissue via an exposed tip 5. The return current flows back through the body of the patient to stimulator 3. Once best stimulation position is located by moving needle 1 and wire 2, the wire is removed and a microstimulator is delivered and implanted via same needle. The same system can be used for muscle, nerve and brain stimulation.

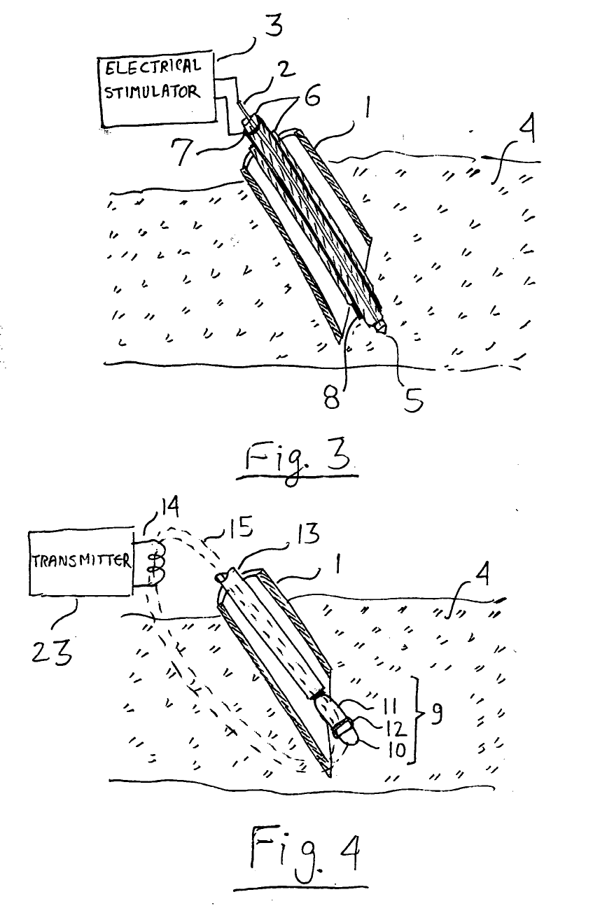

A more accurate stimulation method, closely emulating the microstimulator, is shown in FIG. 3. Two insulated wires, 2 and 7, insulated by insulator 6 are inserted via metallic needle 1 and are terminated with electrodes 5 and 8. The stimulation current from stimulator 3 flows between electrodes 5 and 8. A coaxial configuration of the wires, as shown in FIG. 3, is preferred. Insulation 3 can be made of any dielectric material such as a polymer or ceramic.

FIG. 4 is a cross section showing a microstimulator 9 being delivered via needle 1. While being delivered, the microstimulator can be powered and programmed by electromagnetic coupling 15 to transmitter coil 14 driven by transmitter 23. The details of powering and programming microstimulators are well known in the art and covered by U.S. Pat. No. 5,312,439. Microstimulator 9 is encased in a metallic case made of parts 10 and 11 joined by a non-conductive hermetic seal such as a fused glass to metal seal 12. The way the electromagnetic field 15 penetrates the metal case is explained later in this disclosure. The microstimulator can be used to locate the best implantation site without the use of an external electrical stimulator. It is held in place by rod 13. Rod 13 is also used to implant the device.

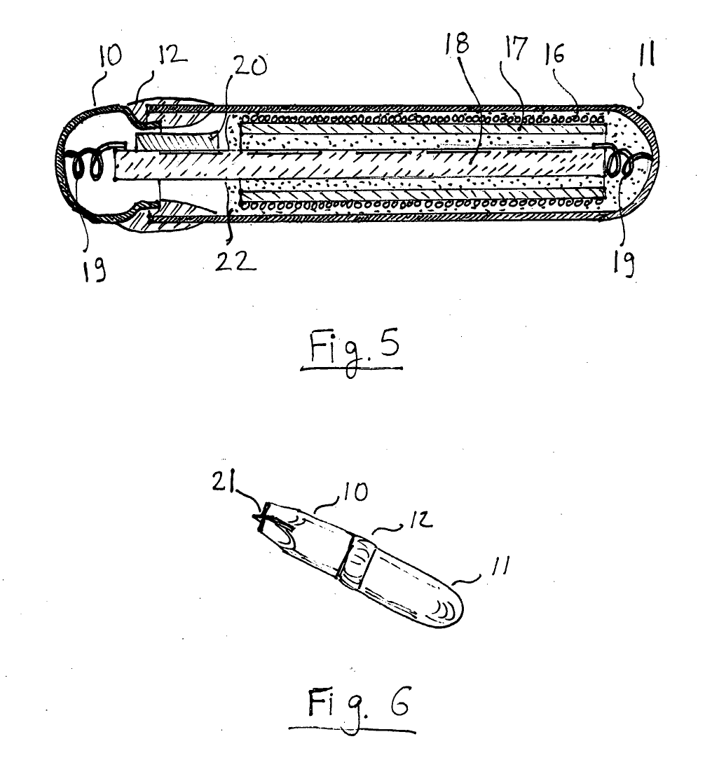

FIG. 5 shows a cross section of a metal encased microstimulator. The case is made of parts 10 and 11, joined by a non-conductive hermetic seal 12. Seal 12 is typically made of low melting point glass or frit. Parts 10 and 11 are made of type 316L stainless steel, titanium, Kovar (an iron-nickel cobalt alloy) or any other suitable metal. Kovar has a thermal expansion coefficient matched to glass seals. When selecting the metal for parts 10 and 11, factors besides biocompatibility have to be considered. One factor is having a coefficient of thermal expansion matched to the sealing material. Another factor is having high resistivity and permeability in order to minimize the attenuation of the electromagnetic field. A good overall combination is type 316L stainless steel, which has well known good biocompatibility. It is also possible to use an enclosure made of one metal, such as Kovar, plated with another metal, such as gold or stainless steel. Hermetic seal 12 has to be electrically insulating because parts 10 and 11 also serve as stimulation electrodes and can not be shorted together. Beneficial medicated coatings, such as drug eluting coating, or beneficial surface finishes such as sandblasting (to promote rapid bonding with tissue) can be used on the outside surfaces. Special coatings, such as carbon, can be used to achieve electrically conductive highly hydrophobic surfaces. Inside the case a coil 16, typically wound on a ferrite tube 17, picks up the transmitted power and commands. A silicon integrated circuit 18 is powered by the power from coil 16 and stores the energy in storage device 20. The storage device can be a capacitor, a super-capacitor or a rechargeable battery. New types of rechargeable batteries, such as nano-tantalate batteries, are particularly suitable because they can be charged and discharged thousands of times. The connection between the integrated circuit 18 and the case parts 10 and 11 can be made via small springs 19. The inside of the microstimulator is at least partially filled by a polymeric or ceramic material 22. After assembly, the seal material 12 can be fused by a flame, laser (such as CO₂) or radiant heat. Because of the low mass, the fusing time is very short and the internal components are not damaged by the heat. An alternate sealing method is shown in FIG. 6. The enclosure, made of parts 10, 11 and 12 is pre-fused. After inserting the electronic assembly into the enclosure, the end is crimped and welded to form a hermetic seal 21. This allows the use of higher temperature sealing material 12, as it is fused before the housing is filled. It was found that by proper selection of the materials for the case, a surprisingly low amount of electromagnetic shielding is produced. The coupling between the coil 16 inside the microstimulator and coil 14 (shown in FIG. 4) located outside is within 1% of the coupling achieved by the prior art of using a glass or silicon enclosure. Metal is much preferred as it can be made thinner, stronger and can be used as stimulation electrodes thus further reducing the size of the microstimulators. Unlike glass or ceramics, metal does not shatter when broken, further eliminating the need for protective coatings.

A partial explanation for this unexpected performance is the well known “skin effect” when using high frequency currents. Such currents do not penetrate the full cross section of the conductor, but travel mainly on the outside “skin”. For a metal having a resistivity ρ and absolute magnetic permeability μ, when using a current of frequency f, most of the current will stay in a layer having a thickness of (ρ/πμ)^{1/2}. The total resistance is proportional to ρ divided by the skin depth: ρ/(ρ/πμ)^{1/2}=(π∥μ)^{1/2}. Compared to the cross section of the copper wire coil 16, the high resistance of the case creates surprisingly little attenuation. For example, if a dielectric case is considered having 100% coupling, the following results were measured for a 25 um thick metal case and over a frequency range of 100 KHz to 10 MHz:

Type 316L Stainless steel: 99% coupling
Brass: 25% coupling
Copper: 10% coupling.

The same effects explain the high coupling when the microstimulator is inside the hypodermic needles. The coupling is about 75% when using a standard 2 mm stainless steel hypodermic needle. The ability to make both the enclosure of the microstimulator and the delivery needle of metal reduces the size of the required delivery needle and increases the strength and safety of the device. A further improvement is the use of the case as an electrode, further reducing size or increasing range for a given size. The two parts of the metal case can serve as two electrodes, or at least one of the parts can serve as an electrode while the second electrode is a wire sealed to the case via a hermetic seal. This may be desired where a larger spacing is required between the electrodes. In such a construction the “two parts of the case” should be interpreted as a case and a separate electrical conductor joined to the case by an electrically insulated hermetic seal. It is well known that the stimulation current should not have a direct current component. The standard way to achieve that is by capacitive coupling to the stimulation electrode but it is also possible to produce a symmetric bipolar drive waveform having no direct current component (i.e. no net charge). Such waveforms can be produced by the integrated circuit inside the microstimulator using the well known H-Bridge circuit, similar to the circuit used for bipolar drive of motors. Another alternative to a coupling capacitor is to coat the case with porous tantalum oxide forming a capacitor.

1. A system for implanting a microstimulator in a tissue comprising of a metal encased microstimulator, a metal hypodermic needle and an insulated wire connected to an electrical stimulator, said wire inserted via said needle prior to implanting said microstimulator via said needle.

2. A system for implanting a microstimulator in a tissue comprising of a metal encased microstimulator, a metal hypodermic needle and two insulated wires connected to an electrical stimulator, said wires inserted via said needle prior to implanting said microstimulator via said needle.

3. A metal encased microstimulator having a hermetic enclosure comprising of two metal parts joined by an electrically insulating hermetic seal, and at least one of said parts forms a case for said microstimulator.

4. A microstimulator as in claim 3 wherein at least one of said metal parts also serves as a stimulation electrode.

5. A microstimulator as in claim 3 wherein at least one of said metal parts has a medicated coating.

6. A microstimulator as in claim 3 wherein at least one of said metal parts is plated by a different material.

7. A microstimulator as in claim 3 wherein at least one of said metal parts is made of gold plated Kovar.

8. A microstimulator as in claim 3 wherein at least one of said metal parts is made of type 316L stainless steel.

9. A microstimulator as in claim 3 also containing an energy storage device.

10. A microstimulator as in claim 3 also containing a bipolar output drive circuit.
